(19) United States
(12) Patent Application Publication
(10) Pub. No.: US 2006/0189543 A1
(43) Pub. Date: Aug. 24, 2006

(54) Compositions and methods for the treatment of leukemia

(76) Inventor: Richard A. Rosenbloom, Elkins Park, PA (US)

Correspondence Address: KNOBLE YOSHIDA & DUNLEAVY, LLC, Eight Penn Center, Suite 1350, 1628 John F. Kennedy Blvd., Philadelphia, PA, 19103, US

(21) Appl. No.: 11/065,532

(22) Filed: Feb. 23, 2005

(51) Int. Cl. A61K 38/05; A61K 31/7048; A61K 31/59; A61K 31/385; A61K 31/353; A61K 31/375; A61K 31/12; A61K 31/07

(52) U.S. Cl. 514/18; 514/167; 514/474; 514/27; 514/688; 514/440; 514/410; 514/725; 514/468; 514/456

(57) ABSTRACT

A method for the treatment of leukemia by internal administration of a composition including one or more compounds that inhibit cytokines and one or more antioxidants, optionally formulated in a pharmaceutically acceptable carrier. The composition of the present invention may further include optional ingredients such as flavonoids, flavonoid derivatives, and compounds that regulate cell differentiation and/or cell proliferation. A method for the internal administration of a composition for the purpose of treating leukemia involves the administration of an effective amount of a composition including one or more compounds that inhibit cytokines and one or more antioxidants to a person who has leukemia. Compositions useful in the method are also disclosed.

### BACKGROUND OF THE INVENTION

1. Field of the Invention

The present invention relates to methods for treating leukemia.

2. Description of the Prior Art

Cancers, including leukemia, are the leading cause of death in animals and humans. The exact cause of many cancers is not known, but links between certain activities such as smoking or exposure to carcinogens and the incidence of certain types of carcinomas, lymphomas, e.g., leukemia and tumors, has been shown by a number of researchers. Additionally, exposure to extensive radiation or exposure to ionizing radiation may cause cancer, particularly leukemia. Some genetic defects (e.g. Down's syndrome, Fanconi's anemia) also predispose certain persons to contracting leukemia.

Leukemia is a chronic or acute progressive, malignant neoplasm of the blood forming organs marked by diffuse replacement of the bone marrow development of leukocytes and their precursors in the blood and bone marrow. This replacement is accompanied by a reduced number of erythrocytes and blood platelets, resulting in anemia and increased susceptibility to infection and hemorrhage. Other typical symptoms include fever, pain in the joints and bones, and swelling of the lymph nodes, spleen and liver. Leukemia is classified clinically on the basis of (1) the duration and character of the disease: acute or chronic; (2) the predominant proliferating cells: myelocytic, granulocytic, or lymphocytic; and (3) increase in or maintenance of the number of abnormal cells in the blood: preleukemic.

Acute leukemia consists of acute lymphobastic leukemia (ALL) and acute myelogenous leukemia (AML). AML can be further subdivided into acute myelocytic leukemia, acute promyelocytic, acute nonlymphocytic, and acute monocytic leukemia. Chronic leukemia consists of chronic lymphocytic leukemia, chronic myelocytic leukemia and chronic myelogenous leukemia. Also included in the definition of leukemia is myelodysplastic syndrome (MDS), which is a group of syndromes that includes preleukemia, refractory anemias, Philadelphia chromosome (Ph)-negative chronic myelocytic leukemia, chronic myelomonocytic leukemia, and (Ph)-negative chronic myelocytic leukemia, chronic myelomonocytic leukemia, and agnogenic myeloid metaplasia. MDS is characterized by clonal proliferation of hematopoietic cells, including erythroid, myeloid and megakaryocytic forms.

Numerous treatments exist to control or treat cancers including leukemia. For example, U.S. Pat. No. 6,759,064 describes compositions using a combination of catechins and vanilliods for the prevention and treatment of cancer. U.S. Pat. No. 6,187,315 describes the use of tannin complexes to treat cancer. U.S. Pat. No. 6,811,795 describes the use of compositions derived from plant material obtained from Glinus lotoides, Ruta chalepensis, Hagenia abyssinica, and/or Millettia ferrginea to treat cancer. U.S. Pat. No. 6,649,648 describes the use of isoflavones in therapeutic compositions to treat cancer.

While many types of chemotherapeutic agents have been shown to be effective against leukemia, not all types of leukemia cells respond to these agents, and, unfortunately, many of these agents also destroy normal cells.

Despite advances in the field of leukemia treatments, the leading therapies to date are radiation, chemotherapy and bone marrow transplants. However, these therapies generally harm normal cells as well as leukemia cells. Ideally, cytotoxic agents that have specificity for leukemia cells while only minimally affecting normal healthy cells would be extremely desirable. Unfortunately, none have been found and instead agents which target especially rapidly dividing cells (both diseased and normal) have been used.

There still remains a need in the art for effective compositions and methods to treat leukemia. It is an objective of certain embodiments of the present invention to provide methods to effectively treat leukemia by internal administration of a composition that, when administered, will treat leukemia.

These and other objects of the present invention will be apparent from the summary and detailed descriptions of the invention, which follow.

### SUMMARY OF THE INVENTION

In a first aspect, the present invention relates to a composition for the treatment of leukemia. The composition for the treatment of leukemia includes a compound that acts as a cytokine-inhibitor, one or more antioxidants and optionally, a pharmaceutically acceptable carrier.

In a second aspect, the present invention relates to a method of internally administering a composition for the treatment of leukemia. In the method, an effective amount of a suitable composition is internally administered to a mammal with leukemia to treat said leukemia. The internally administered composition for the treatment of leukemia includes a compound that acts as a cytokine-inhibitor, one or more antioxidants and optionally, a pharmaceutically acceptable carrier.

### DETAILED DESCRIPTION OF THE PREFERRED EMBODIMENTS

In a first aspect, the present invention relates to a composition for the treatment of leukemia. The composition for the treatment of leukemia includes a compound that acts as a cytokine-inhibitor, one or more antioxidants and optionally a pharmaceutically acceptable carrier.

In a second aspect, the present invention relates to a method for internal administration of a composition for the treatment of leukemia. In this aspect of the invention, the internally administered composition includes a mixture of a compound that acts as a cytokine inhibitor, one or more antioxidants and, optionally, a pharmaceutically acceptable carrier.

One ingredient in the composition for the treatment of leukemia is a compound that acts as a cytokine inhibitor, i.e. has cytokine-inhibiting properties. Such compounds include turmeric and its extracts, particularly the curcuminoids such as curcumin. The curcuminoids have been reported to inhibit NF-kappa B activity as well as inhibiting tumor necrosis factor (TNF)-mediated adhesion of monocytes to endothelial cells. The curcuminoids may also inhibit interleukins (IL) particularly IL-12. Additionally, curcuminoids inhibit various kinases, synthases and transferases in lymphocytes and macrophages. Curcuminoids have been reported to be protease-inhibitors as well as inhibitors of proteasomal functions. In addition, the curcuminoids have antioxidant activity. Without being restricted to a particular mode of action, the extracts of turmeric and/or the curcuminoids used in the present invention are included for their cytokine-inhibiting activity and thus the compositions of the present invention contain an amount of cytokine-inhibition composition to provide cytokine-inhibiting activity.

Exemplary curcuminoids include but are not limited to curcumin (diferuloylmethane), desmethoxycurcumin (hydroxycinnamoyl feruloylmethane), and bis-desmethoxycurcumin (dihydroxydicinnamoyl methane) (see Drug Analysis by Chromatography and Microscopy, p. 169, Ann Arbor Science Inc., 1973), which may be purchased from commercial sources or obtained from turmeric. Methods for isolating curcuminoids from turmeric are known, (see Janaki and Bose, An Improved Method for the Isolation of Curcumin From Turmeric, J. Indian Chem. Soc. 44:985 (1967)). Alternatively, curcuminoids for use in the present invention can be prepared by synthetic methods.

Another ingredient in the composition for the treatment of leukemia is an antioxidant. The antioxidant may be a single compound or material or a mixture of two or more compounds and/or materials. Compounds and materials which may be used as antioxidants are those which exhibit antioxidant activity when administered to a patient without causing severe adverse side affects when used in an amount effective to provide sufficient antioxidant activity, and which do not react with one or more of the ingredients of the composition resulting in a substantial loss of activity of one or more of the ingredients. Preferred antioxidants are those that occur naturally in the human body and/or materials obtained from plants or animals, or derivatives thereof.

Preferred antioxidants are selected from ascorbic acid (vitamin C) and its esters, for example, ascorbyl palmitate; vitamin A and its esters, for example, vitamin A palmitate; vitamin E and its esters, for example, vitamin E acetate; α-lipoic acid, especially DL-α-lipoic acid; carotenoids such as β-carotene; chlorophyllin and its salts; coenzyme Q10; glutathione; green tea polyphenols, such as (−)-epigallocatechin-3-gallate; catechin; galangin; rutin; luteolin; morin; fisetin; silymarin; apigenin; gingkolides; hesperitin; cyanidin; citrin and structurally similar derivatives thereof which exhibit antioxidant activity. Even more preferably, mixtures of two or more antioxidants are employed in the composition of the present invention.

The antioxidants may also be used in the form of their pharmaceutically acceptable salts and this may be preferred in some cases to increase solubility or dispersability, to reduce adverse side effects, etc.

In another aspect, the antioxidant used in the composition of the present invention may include one or more antioxidant enzymes. The antioxidant enzymes useful in the present invention are those capable of scavenging radicals, promoting radical scavengers or preventing radical formation. The preferred antioxidant enzymes useful in the present invention include superoxide dismutase, catalase, glutathione peroxidase and methionine reductase. Other antioxidant enzymes with activities similar to those mentioned explicitly above, may also be used. In addition, one or more of the antioxidant enzymes may act in combination with one or more of the antioxidant compounds in the composition to, for example, scavenge free radicals and/or prevent cell damage in the skin.

The antioxidant component of the composition is used in an amount effective to provide significant antioxidant activity when systemically administered to a patient at an acceptable daily dose rate.

The antioxidants used in the composition of the present invention are preferably selected not only for their antioxidant activity, but also based on other beneficial effects that particular compounds may provide. For example, a racemic mixture of α-lipoic acid not only has a strong antioxidant activity but also has a recycling effect on vitamins C and E, and thus is a suitable antioxidant for the present invention. In addition, α-lipoic acid can function in both lipid and non-lipid environments. Similarly, vitamin E and its esters may contribute to an anti-cancer effect and may have beneficial effects on the skin and is thus is also a preferred antioxidant. Vitamin C and its esters are not only antioxidants, but also exhibit a strong combinatorial effect with vitamin E and its esters when used together. In fact, vitamin E and its esters, and vitamin C and its esters can mutually reinforce one another by a mechanism in which one antioxidant (reducing agent) acts as a regenerator for the oxidized form of the other. Vitamin A (retinol or retinyl ester) may also have anti-cancer effects.

Another antioxidant that can be employed in the composition of the present invention is green tea polyphenol or green tea extract, which contains compounds such as (−)-epigallocatechin-3-gallate, (−)-epigallocatechin-3-gallate, (−)-epigallocatechin and/or (−)-epicatechin. Studies (see Elmets, C. A. et al, J. Am. Acad. Dermatol., 44 (3); 425-32, March, 2001) have shown that green tea polyphenol or extract is effective in inhibiting erythema and preventing Langerjans cells from some forms of ultraviolet radiation damage.

A preferred antioxidant for use in the composition of the present invention includes chlorophyllin and/or its salts. Chlorophyllin and/or its salts may be included in the composition of the present invention in amounts, which, when administered to a patient according to a method of the present invention, provide a daily dosage of about 20 milligrams to about 540 milligrams. Chlorophyllin and/or its salts may be an alfalfa extract or extracted from silkworm feces. Chlorophyllin and/or its salts may also be purchased from common commercial sources such as Aldrich Chemical Company.

In some embodiments a mixture of both chlorophyllin and α-lipoic acid may be employed.

Optionally, the composition of the present invention includes one or more flavonoids and/or flavonoid derivatives, flavonones, flavones, flavonols, flavan-3-ols and/or anthocyanidins. Certain flavonoids known to have antioxidant activity may be employed as all or part of the antioxidant component of the compositions of the present invention. Alternatively, flavonoids may be included as an additional ingredient in the compositions of the invention.

Exemplary flavonoids and flavonoid derivatives include 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; and wogonin.

The most preferred flavonoids and/or flavonoid derivatives are quercetin, quercetrin, myricetin, kaempferol and myrecetrin. Also, pharmaceutically acceptable salts of these flavonoids and/or flavonoid derivatives may be employed. The particular flavonoids and/or flavonoid derivative included in the composition may be determined by factors such as toxicity, bioavailability, solubility or dispersability, and antioxidant activity, among others. The particular flavonoids and/or flavonoid derivatives mentioned above are also preferred since some of these compounds may provide additional beneficial effects in the composition of the present invention. For example, quercetin may also have an antioxidative and anticlastogentic effect.

In embodiments that utilize flavonoids and/or flavonoid derivatives, an effective amount of the composition for each daily administration contains about 0.01 grams to about 2 grams of flavonoids and/or flavonoid derivatives. More preferably, the flavonoids and/or flavonoid derivatives are employed in the composition in an amount of about 0.05 to about 1 gram, and, most preferably, the flavonoids and/or flavonoid derivatives are employed in an amount of 0.1 to about 0.4 grams in the composition.

Optionally, a compound that regulates cell differentiation and/or cell proliferation may be added to the composition. The compound that regulates cell differentiation and/or cell proliferation may be selected from suitable compounds that have this activity. Suitable compounds that regulate cell differentiation and/or cell proliferation are those that do not induce significant, adverse side effects when administered to a patient in amounts that regulate cell differentiation and/or cell proliferation, and which do not react with one or more of the ingredients of the composition resulting in a substantial loss of activity of one or more of the ingredients. Preferred compounds for regulating cell differentiation and/or cell proliferation are those that occur naturally in the human body and/or materials obtained from plants or animals which may be administered to humans without significant, adverse side effects in the amounts used, or derivatives thereof.

More preferably, the compounds that regulate cell differentiation and/or cell proliferation used in the present invention inhibit or prevent cell differentiation or cell proliferation. Even more preferably, the compounds that regulate cell differentiation and/or cell proliferation used in the present invention accomplish at least one of the following: maintenance of cellular homeostasis and normal cell metabolism, regulation of cell differentiation, induce certain cancer cells to differentiate into normal cells, preferably by working in combination with vitamin A, maintenance of the epidermal permeability barrier, inhibition of cancer cell differentiation, and inhibition of cancer cell proliferation.

Exemplary compounds that regulate cell differentiation and/or cell proliferation are vitamin D₃, vitamin D₃ analogs, compounds that may be converted or metabolized into vitamin D₃ in the human body, and metabolites thereof. Exemplary compounds that may be converted or metabolized into vitamin D₃ include common cholesterols illustrated below. The cholesterol illustrated below may be converted into Provitamin D when a hydrogen is removed from the number 7 carbon, which then forms a double bond with the number 8 carbon, in the second, or ‘B’ ring of the cholesterol molecule. The cholesterol is ‘oxidized’ (that is, an electron is removed with the hydrogen atom), so that the double bond is a consequence of 2 mutually shared electrons between carbons 7 and 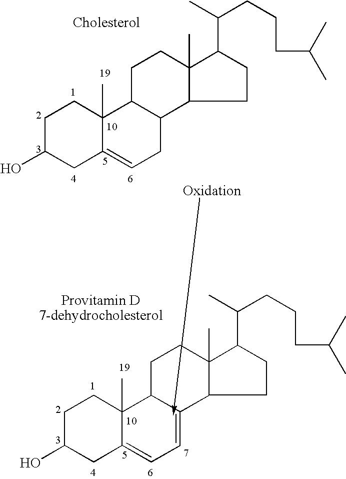
Provitamin D may be converted to Vitamin D₃ by the action of ultraviolet light through human skin. In this reaction, the B ring of the sterol molecule is opened. 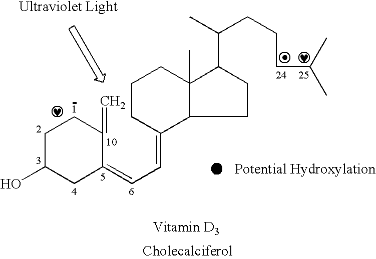
Cholecalciferol, which is Vitamin D₃, may be further converted into another vitamin D intermediate, 25-hydroxycholecalciferol, in the liver by mitochondrial hydroxylase, in the presence of NADPH, and molecular oxygen. 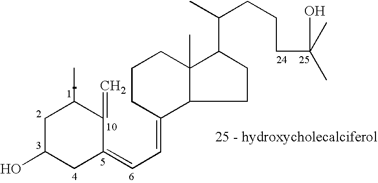
When more active vitamin D₃ is required, 25-hydroxycholecalciferol is transported to the kidney where a new hydrolase enzyme is synthesized. This enzyme introduces another hydroxyl group at position 1, and the bioactive form of Vitamin D₃, calcitriol, is produced.

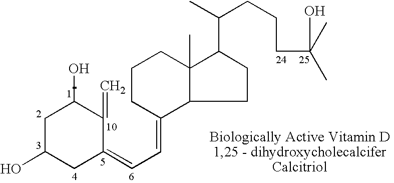

Exemplary vitamin D₃ analogs include 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene. Exemplary vitamin D₃ metabolites include 1,25-dihydroxyvitamin D₃. Also, pharmaceutically acceptable salts of the compounds that regulate cell differentiation and/or cell proliferation may be employed. The most preferred compound that regulates cell differentiation and/or cell proliferation is vitamin D₃.

The compound that regulates cell differentiation and/or cell proliferation is used in an amount effective to regulate cell differentiation and/or cell proliferation when internally administered to a patient suffering from leukemia.

The internal administration routes that may be employed to administer the composition of the present invention include, but are not limited to, oral, parenteral, intravenous (IV), intramuscular (IM), rectal, buccal, nasal, or any other parenteral route (i.e. a route that does not involve the alimentary canal) such as subcutaneous, intrasternal, intraperitoneal or subabdominal.

The maximum daily dosage of certain of the active ingredients should not exceed 8000 mg of curcuminoids (containing at least 5% curcumin), 540 mg of sodium copper chlorophyllin, and 1200 mg of alpha lipoic acid. Preferred individual daily dosages in accordance with the present invention can be administered multiple times per day, or in a quantity sufficient for one daily dose and should not exceed the maximum daily allowable dosage.

In general, the total daily dose ranges of the active cytokine inhibitor for the conditions described herein are generally from about 1-1000 mg and from about 1-1000 mg of antioxidant. A preferred total daily dose is from about 25-300 mg of the cytokine-inhibitor and from about 100-500 mg of the antioxidant. Maximum total daily dose should not exceed 8000 mg of the cytokine-inhibitor, and 1200 mg of the antioxidant.

Preferably the cytokine-inhibitor and antioxidant formulation of the invention is given daily until remission, followed by two to ten additional cycles, each lasting about 60 days in duration. When the dose is administered orally, a sustained release formulation is preferred so that a fairly constant level of the cytokine-inhibitor and the antioxidant is provided over the course of treatment, which is generally at least 48 hours and preferably at least 96 hours per cycle. As the cytokine-inhibitor and antioxidant are not particularly toxic, the formulation may be administered for as long as necessary to achieve the desired therapeutic effect.

An exemplary course of treatment of a patient with leukemia can involve administration by intravenous infusion of the cytokine-inhibitor and the antioxidant in an aqueous solution at a daily dose of about 1-8000 mg of the cytokine-inhibitor and from about 1-1200 mg of the antioxidant, based on a 70 kg adult patient, for a period of several days. The course of treatment may be repeated for up to ten times over approximately 10 months with a break of about three to six weeks in between courses. The post-remission course of treatment involves infusion of the cytokine-inhibitor at a daily dose of about 1-8000 mg and from about 1-1200 mg of antioxidant per kg of body weight of the patient on a daily or weekdays-only basis for a cumulative total of 25 days.

The preferred individual doses should contain from about 1 to about 1000 mg of cytokine-inhibiting compound(s), such as curcuminoids (containing at least 5% curcumin), from about 1 to about 1200 mg of antioxidant. In a particularly preferred composition, the antioxidant may contain up to about 540 mg of sodium copper chlorophyllin, and from about 0 to about 1000 mg of alpha lipoic acid. More preferably, the individual doses will contain from about 5 to about 200 mg of curcuminoids, about 50 to about 500 mg of sodium copper chlorophyllin, and from about 50 to about 300 mg of alpha lipoic. More preferably, the individual doses will contain from about 25 to about 75 mg of curcuminoids, about 100 to about 300 mg of sodium copper chlorophyllin, and from about 75 to about 225 mg of alpha lipoic acid.

The composition may be administered 1-10 times per day, as needed, more preferably, 2-6 times per day, as needed, or most preferably, 3 times per day, up to the maximum daily dosage, to a person with leukemia. Preferably, during each oral administration of a dose, a person ingests 1-5 tablets, capsules, lozenges, or equivalents thereof. More preferably, a person ingests 1-2 tablets, capsules, lozenges or equivalents during each administration of a dose. Most preferably, the tablets, capsules or lozenges or equivalents thereof are ingested with a fluid such as water, juice, milk, or other suitable fluids.

In a preferred aspect, the method of the present invention involves the oral administration of a composition to a human that has leukemia. The effective amount of the oral composition to be administered will vary depending on such factors as the person being treated, the particular mode of administration, the activity of the particular non-carrier ingredients employed, the age, bodyweight, general health, sex and diet of the person, the time of administration, the rate of excretion, the particular combination of ingredients employed, the total content of the non-carrier ingredients of the oral composition, and the severity of the leukemia. It is within the skill of the person of ordinary skill in the art to account for these factors to provide a suitable dosage and treatment regimen for a standard 70 kg adult, described below. In one embodiment, the compositions of the present invention may be formulated in any acceptable oral dosage forms including, but not limited to, capsules, tablets, lozenges, troches, hard candies, powders, sprays, elixirs, syrups, and suspensions or solutions.

The oral compositions of the present invention are preferably formulated with a pharmaceutically acceptable carrier. The pharmaceutically acceptable oral carrier may include, but is not limited to: (a) carbohydrates including fructose, sucrose, sugar, dextrose, starch, lactose, maltose, maltodextrins, corn syrup solids, honey solids, commercial tablet compositions including Emdex®, Mor-Rex®, Royal-T®, Di-Pac®, Sugar-Tab®, Sweet-Rex®, New-Tab®, (b) sugar alcohols including mannitol, sorbitol, xylitol, and (c) various relatively insoluble excipients including dicalcium phosphate, calcium sulfate, calcium carbonate, microcrystalline cellulose and other pharmaceutical tableting ingredients. Additionally, any of the sugar-free sweeteners commercially available may be used in place of the above listed carbohydrates.

In the case of tablets, for oral use, the pharmaceutically acceptable oral carrier may further include lactose and corn starch. Lubricating agents may also be added to the tablets, including, for example, magnesium stearate, sodium lauryl sulfate and talc. Tablets may also contain excipients such as sodium citrate, calcium carbonate and calcium phosphate. Disintegrants such as starch, alginic acid and complex silicates, may also be employed. Tablets may also include binding agents such as polyvinylpyrrolidone, gelatin, PEG-8000 and gum acacia.

In the case of lozenges for oral use, the common pharmaceutically acceptable oral carrier may further include a binder such as PEG-8000. Preferably lozenges are made in a 0.1 to 15 grams size to allow a suitable dissolution rate for lozenges. More preferably lozenges are made in a 1 to 6 gram size to allow a suitable dissolution rate for lozenges. Dissolution time should be about 15 minutes in water bath testers at 37° C. degrees or about 30 minutes when orally dissolved as lozenges for treating a sore throat, congestion, laryngitis and mucous membrane inflammation.

To directly make compressible lozenges, add the active ingredients to PEG-8000 processed fructose; or add the active ingredient of the composition to crystalline fructose and commercially available, sweet, direct compression products such as Mendell's Sugartab®, Sweetrex®, or Emdex® and add saccharin if desired, flavors as desired, glidants such as silica gel as needed, and lubricants such as magnesium stearate, as needed. The mixture should be kept dry and tableted soon after mixing. The ingredients are mixed and directly compressed into lozenges using conventional pharmaceutical mixing and tableting equipment. The compressive force is preferably sufficient to produce maximum hardness throughout the lozenges, to preserve the dissolution rate, and to maximize the efficacy of lozenges. Dissolution should occur over a sustained period of time of about 5 to 60 minutes, and preferably about 20 to 30 minutes. The composition should be stored in an airtight container and in a cool dark place. Tablets and troches can be manufactured using procedures similar to that described above with minor changes in the optional ingredients.

Alternatively, the oral composition of the present invention may be formulated in liquid form, such as syrups, mouthwashes or sprays with a solvent or dispersant such as water, or other liquids in a pharmaceutically acceptable oral carrier for delivery of the composition to a patient.

The oral composition may also be formulated in chewable compositions such as soft candy, gum drops, liquid filled candies, chewing gum bases and dental supplies, such as toothpastes and mouthwashes by further including fructose, sucrose, or saccharin in the composition, as needed.

The oral composition of the invention may be formulated in capsule form with or without diluents. For capsules, useful diluents include lactose and dried cornstarch. When suspensions are employed, emulsifying and/or suspending agents may be employed in the suspensions. In addition, solid compositions including one or more of the ingredients of the lozenges described above may be employed in soft and hard gelatin capsules.

Preparations for oral administration may be suitably formulated to give controlled release of the active compound. In a preferred embodiment, the compounds of the present invention are formulated as controlled release powders of discrete micro-particles, which can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to, those described in column 3, line 46-column 4, line 8 of U.S. Pat. No. 5,354,556.

The compositions of the present invention may also be formulated into a nasal aerosol or inhalant. Such compositions may be prepared using well-known techniques. For these types of formulations, suitable carriers may include the following ingredients: saline with one or more preservatives, absorption promoters to enhance bioavailability, fluorocarbons and/or other conventional solubilizing or dispersing agents.

In general, the non-carrier ingredients described above, which may include the cytokine-inhibitor, the antioxidant and the optional ingredients, make up from about 0.5-50% by weight of the total composition. Preferably, the non-carrier ingredients will make up about 1-20% by weight of the total composition. More preferably, the non-carrier ingredients make up about 2-10% by weight of the total composition.

The invention further provides an effective amount of a mixture of at least two ingredients, a cytokine-inhibitor and an antioxidant that are formulated as sustained release compositions. As used herein, the term “sustained release formulation” refers to any composition that provides slow, controlled, and/or timed release of one or more active ingredients. In one aspect, a cytokine-inhibitor is formulated as a sustained release and is adjunctively administered with an antioxidant. In another aspect, the effective amount of the antioxidant is formulated as a sustained release formulation and is adjunctively administered with the cytokine-inhibitor. In yet another aspect, both a cytokine inhibitor and an antioxidant are formulated as sustained release formulations or as a single sustained release formulation.

It is understood that the cytokine-inhibitor and the antioxidant levels are maintained over a certain period of time as is desired and can be easily determined by one of skill in the art using this disclosure and available pharmaceutical compendia. In one aspect of the invention, a unique feature of administration comprising a sustained release formulation is included so a constant level of the cytokine-inhibitor and the antioxidant between 48 to 96 hours in the sera.

Such sustained and/or timed release formulations may be made by sustained release means or delivery devices that are well known to those of ordinary skill in the art, such as those described in U.S. Pat. Nos. 3,845,770, 3,916,899, 3,536,809, 3,598,123, 4,008,719, 4,710,384, 5,674,533, 5,059,595, 5,591,767, 5,120,548, 5,073,543, 5,639,476, 5,354,556, and 5,733,566, the disclosures of which are each incorporated herein by reference. These compositions can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the compositions of the invention. Thus, single unit dosage forms suitable for oral administration, such as, but not limited to, tablets, capsules, gelcaps, caplets, powders, and the like, that are adapted for sustained release are encompassed by the present invention.

In an aspect of the invention, the sustained release formulation contains active ingredients such as, but not limited to, microcrystalline cellulose, maltodextrine, ethylcellulose, and magnesium stearate. In yet another highly preferred embodiment, the formulation is synthesized with a CapsuDar™ SR (Biodar, Yavne, Israel) microencapsulation, which consists of the active ingredients microcrystalline cellulose, maltodextrine, ethylcellulose, and magnesium stearate.

As described above, all known methods for encapsulation which are compatible with the properties of the cytokine-inhibitor and the antioxidant are encompassed by this invention. The sustained release formulation is encapsulated by coating particles or granules of the composition of the invention with varying thicknesses of slowly soluble polymers or by microencapsulation. In an aspect of the invention, the sustained release formulation is encapsulated with a coating material of varying thickness (e.g., about 1 micron to 200 microns) that allows the dissolution of the pharmaceutical composition about 48 hours to about 72 hours after administration to a mammal. In another aspect, the coating material is a food-approved additive. In yet another aspect, the coating material is sold under the trademark Eudragit RS or RL (Rohm Pharma, Germany).

In another aspect, the sustained release formulation is a matrix dissolution device, which is prepared by compressing the drug with a slowly soluble polymer carrier into a tablet. In one preferred aspect, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, have a relatively high percentage of plasticizer in the coating in order to permit sufficient flexibility to prevent substantial breakage during compression. The specific amount of plasticizer varies depending on the nature of the coating and the particular plasticizer used. The amount may be readily determined empirically by testing the release characteristics of the tablets formed. If the medicament is being released too quickly, then more plasticizer is used. Release characteristics are also a function of the thickness of the coating. When substantial amounts of plasticizer are used, the sustained released capacity of the coating diminishes. Thus, the thickness of the coating may be increased slightly to make up for an increase in the amount of plasticizer. Generally, the plasticizer in such an embodiment will be present in an amount of about 15 to 30 percent of the sustained release material in the coating, preferably 20 to 25 percent and the amount of coating will be from 10 to 25 percent of the weight of active material, preferably 15 to 20 percent. Any conventional pharmaceutically acceptable plasticizer may be incorporated into the coating.

The levels of circulating cytokine-inhibitor and the antioxidant compositions must be maintained above some minimum therapeutic dose to reduce the number of leukemia cells. In one aspect of the invention, the reduction in the number of leukemia cells is a result of cell death or apoptosis. In another aspect, the reduction in the number of leukemia cells is a result of inhibition of cell growth. In yet another aspect, the reduction in the number of leukemia cells is a result of cell growth arrest.

The sustained release formulations of the invention are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

Various inducers, for example pH, temperature, enzymes, water, or other physiological conditions or compounds, may stimulate the sustained release of an active ingredient. The term “sustained release component” in the context of the present invention is defined herein as a compound or compounds, including, but not limited to, polymers, polymer matrices, gels, permeable membranes, liposomes, microspheres, or the like, or a combination thereof, that facilitates the sustained release of the active ingredient.

The compositions of the invention may be formulated for parenteral administration, e.g., by intramuscular injections or implants for subcutaneous tissues and various body cavities and transdermal devices.

Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In one aspect, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil solutions and suspensions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachnis, maize, almond, cottonseed, and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

The compositions may also be formulated in rectal compositions such as suppositories or retention enemas, e.g, containing conventional suppository bases such as cocoa butter or other glycerides.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of a cytokine-inhibitor and antioxidant in pharmaceutically acceptable form. The cytokine-inhibitor and the antioxidant composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another aspect, a kit of the invention further comprises a needle or syringe, preferably packaged in sterile form, for injecting the complex, and/or a packaged alcohol pad. Instructions are optionally included for administration of the cytokine-inhibitor and the antioxidant composition by a clinician or by the patient.

In another aspect of the invention, the magnitude of a therapeutic dose of the active ingredients in the acute or chronic management of leukemia will vary with the severity of the condition to be treated and the route of administration. The dose, and dose frequency, will also vary according to the age, body weight, condition and response of the individual patient, and the particular active ingredient combination used. All combinations described in the specification are encompassed as therapeutic, active ingredients and mixtures and it is understood that one of skill in the art would be able to determine a proper dosage of particular cytokine-inhibitor and antioxidant mixtures using the parameters provided in the invention. Furthermore, one of ordinary skill in the art would be able to vary the dose of the cytokine-inhibitor relative to the amounts of the antioxidant present, based on the guidance provided throughout the invention.

In addition, the cytokine-inhibitor and antioxidant carrier could be delivered via charged and uncharged matrices used as drug delivery devices such as cellulose acetate membranes, also through targeted delivery systems such as fusogenic liposomes attached to antibodies or specific antigens.

In practical use, the cytokine-inhibitor and the antioxidant can be combined as the active ingredient(s) in intimate admixture with a pharmaceutical carrier according to conventional pharmaceutical compounding techniques. The carrier may take a wide variety of forms depending on the form of preparation desired for administration, e.g., oral or parenteral (including tablets, capsules, powders, intravenous injections or infusions). In preparing the compositions for oral dosage form any of the usual pharmaceutical media may be employed, e.g., water, glycols, oils, alcohols, flavoring agents, preservatives, coloring agents, and the like; in the case of oral liquid preparations, e.g., suspensions, solutions, elixirs, liposomes and aerosols; starches, sugars, micro-crystalline cellulose, diluents, granulating agents, lubricants, binders, disintegrating agents, and the like in the case of oral solid preparations e.g, powders, capsules, and tablets. In preparing the compositions for parenteral dosage form, such as intravenous injection or infusion, similar pharmaceutical media may be employed, e.g., water, glycols, oils, buffers, sugar, preservatives and the like know to those skilled in the art. Examples of such parenteral compositions include, but are not limited to Dextrose 5% (w/v), normal saline or other solutions. The volume of dilution fluid will vary according to the total dose administered and over the length of the period of time of administration.

In an alternative aspect of the invention, the effect of the therapeutic cytokine-inhibitor and antioxidant on leukemia treatment can be monitored by any methods known in the art, including but not limited to monitoring leukocytes in patient sera and bone marrow biopsies. Desirable blood levels may be maintained by a continuous infusion of cytokine inhibitor and antioxidant as ascertained by plasma levels. It should be noted that the attending physician would also know how to and when to adjust treatment to higher levels if the clinical response is not adequate (precluding toxic side effects, if any).

The foregoing detailed description of the invention is not intended to limit the scope of the invention in any way and should not be construed as limiting the scope of the invention. The scope of the invention is to be determined from the claims appended hereto.

### EXAMPLE 1

Four examples of formulations for use in the systemic administration of patients are provided.

Table 1 is an example of a Base. Table 1 below shows an exemplary unit dose for a Base Formulation. The Base Formulations may be systemically administered up to 7 times per a day.

TABLE 1

| Ingredients | Concentation |
|---|---|
| Turmeric (5% curcumin) | 50 mg |
| Sodium Copper Chlorophyllin | 75 mg |
| Excipients | |

Table 2 below shows the formulation “Option 1.” Option 1 has the active primary ingredients plus additional ingredients. Table 2 shows an exemplary unit dose for Option 1. Option 1 may be systemically administered up to 6 times per day.

TABLE 2

| Ingredients | Concentration |
|---|---|
| Turmeric (5% curcumin) | 50 mg |
| Sodium Copper Chlorophyllin | 75 mg |
| Alpha Lipoic Acid | 200 mg |
| Vitamin E tocopherol | 30 mg |
| Glutathione | 5 mg |
| Excipients | |

Table 3 below shows the formulation “Option 2.” Option 2 has the active primary ingredients plus additional ingredients. Table 3 shows an exemplary unit dose for Option 2. Option 2 may be systemically administered up to 7 times per a day.

TABLE 3

| Ingredients | Concentration |
|---|---|
| Turmeric (5% curcumin) | 50 mg |
| Sodium Copper Chlorophyllin | 75 mg |
| Quercetin | 250 mg |
| Vitamin C | 20 mg |
| Excipients | |

Table 4 below shows the formulation for Option 3. Option 3 has the active primary ingredients plus alternate ingredients. Table 4 shows an exemplary unit dose for this formulation. Option 3 is to be systemically administered up to 7 times per a day.

TABLE 4

| Ingredients | Concentration | |
|---|---|---|
| Turmeric (5% curcumin) | 50 | mg |
| Sodium Copper Chlorophyllin | 75 | mg |
| Vitamin A | 10,000 | IU |
| Excipients | | |

1. A method for the treatment of leukemia comprising the step of internally administering to a human with leukemia, a composition which comprises an amount of one or more compounds selected from the group consisting of curcumin (diferuloylmethane), desmethoxycurcumin (hydroxycinnamoyl feruloylmethane), and bis-desmethoxycurcumin (dihydroxydicinnamoyl methane), which is effective, when administered internally, to inhibit at least one cytokine, and an effective amount of one or more antioxidants selected from the group consisting of ascorbic acid, dehydroascorbic acid, ascorbic acid esters, Ester-C® vitamin C nutritional supplement, vitamin A, esters of vitamin A, vitamin E, esters of vitamin E, α-lipoic acid, carotenoids, chlorophyllin, coenzyme Q10, glutathione, green tea polyphenols, glutathione, galangin, rutin, luteolin, morin, fisetin, silymarin, apigenin, gingkolides, hesperitin, cyanidin, citrin, aldonic acids, aldono-lactones, aldono-lactides, pharmaceutically acceptable salts of each of the foregoing antioxidants, superoxide dismutase catalase, glutathione peroxidase and methionine reductase.

2. The method as claimed in claim 1, wherein the composition further comprises at least one compound that inhibits cell differentiation and cell proliferation selected from the group consisting of vitamin D₃, 1(S), 3(R)-dihydroxy-20(R)-(1-ethoxy-5-ethyl-5-hydroxy-2-heptyn-1-yl)-9,10-seco-pregna-5(Z), 7(E), 10(19)-triene, cholesterols, 7-dehydrocholestrol, 1,25-dihydroxyvitamin D₃, and 25-hydroxycholecalciferol, calcitriol, metabolites thereof, and pharmaceutically acceptable salts thereof.

3. The method as claimed in claim 2, wherein the one or more compounds that inhibit at least one of cell differentiation and cell proliferation is vitamin D₃.

4. The method as claimed in claim 1, wherein the one or more antioxidants are selected from the group consisting of: ascorbyl palmitate, ascorbic acid, vitamin A, vitamin A ester, vitamin E, vitamin E ester, α-lipoic acid, carotenoid, chlorophyllin, chlorophyllin salt, coenzyme Q10, glutathione, galangin, rutin, luteolin, morin, fisetin, silymarin, apigenin, gingkolides, hesperitin, cyanidin, citrin and pharmaceutically acceptable salts thereof.

5. The method as claimed in claim 1, wherein the antioxidant comprises one or more antioxidants selected from the group consisting of superoxide dismutase catalase, glutathione peroxidase and methionine reductase.

6. The method as claimed in claim 1, wherein the composition further comprises at least one flavonoid or flavonoid derivative selected from the group consisting of: 1,2,3,6-tetra-o-gallyol-o-β-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; and wogonin.

7. The method as claimed in claim 6, wherein the flavonoids and flavonoid derivatives are selected from the group consisting of: quercetin, quercetrin, myricetin, kaempferol and myrecetrin.

8. The method as claimed in claim 1, wherein the composition comprises chlorophyllin.

9. The method as claimed in claim 1, wherein the composition comprises α-lipoic acid.

10. The method as claimed in claim 1, wherein the composition comprises curcumin.

11. The method as claimed in claim 1, wherein about 1 to about 8000 mg of curcuminoids, an amount of chlorphyllin present in about 1 to about 540 mg of sodium copper chlorophyllin, and from about 0 to about 1200 mg of alpha lipoic acid are administered per day.

12. The method as claimed in claim 11 wherein about 5 to about 200 mg of curcuminoids, an amount of chlorophyllin present in about 50 to about 500 mg of sodium copper chlorophyllin, and about 50 to about 300 mg of alpha lipoic acid are administered per day.

13. The method as claimed in claim 12, wherein about 25 to about 75 mg of curcuminoids, an amount of chlorophyllin present in about 100 to about 300 mg of sodium copper chlorophyllin, and about 75 to about 225 mg of alpha lipoic acid are administered per day.

14. The method as claimed in claim 1, wherein the composition is administered orally, intravenously, intramuscularly, intra-rectally, intra-nasally, subcutaneously, intra-sternally or by sub-abdominal administration.

15. The method of claim 1, wherein each individual dose of the composition comprises about 1-1000 mg of a cytokine-inhibitor and about 1-1200 mg of one or more antioxidants.

16. The method of claim 1, wherein the cytokine-inhibiting composition comprises curcuminoids and the antioxidant comprises chlorophyllin.

17. The method of claim 16, wherein the antioxidant further comprises alpha lipoic acid.

18. A composition for the treatment of leukemia comprising about 1 to about 1000 mg of curcuminoids, an amount of chlorophyllin present in about 1 to about 540 mg of sodium copper chlorophyllin, and about 1 to about 1000 mg of alpha lipoic acid.

19. A composition for the treatment of leukemia comprising about 5 to about 200 mg of curcuminoids, an amount of chlorophyllin present in about 50 to about 500 mg of sodium copper chlorophyllin, and about 50 to about 300 mg of alpha lipoic acid.

20. A composition for the treatment of leukemia comprising about 25 to about 75 mg of curcuminoids, an amount of chlorophyllin present in about 100 to about 300 mg of sodium copper chlorophyllin, and about 75 to about 225 mg of alpha lipoic acid.
